(19) United States
(12) Patent Application Publication
(10) Pub. No.: US 2024/0041185 A1
(43) Pub. Date: Feb. 8, 2024

(54) COSMETIC APPLICATOR

(71) Applicant: NATCH CORPORATION CO., LTD, Koshigaya-shi, Saitama (JP)

(72) Inventor: Yasuhiro JONO, Koshigaya-shi, Saitama (JP)

(21) Appl. No.: 18/011,598

(22) PCT Filed: Aug. 5, 2022
(86) PCT No.: PCT/JP2022/030168

§ 371 Date: Dec. 20, 2022

(30) Foreign Application Priority Data
Oct. 13, 2021 (JP) 2021-167838
Feb. 15, 2022 (JP) 2022-021115

(51) Int. Cl. A45D 40/20

(52) U.S. Cl. CPC A45D 40/205; A45D 2040/208; A45D 2200/1072

(57) ABSTRACT

A cosmetic applicator (1) comprises a spiral rod (7) formed with a spiral groove (16) on an inner periphery of a column-shaped hollowed portion, a lead chuck member (5) including a reciprocating bar (9) which has a fitting portion fitted in the spiral groove (6) and which is able to be reciprocated from through a tip end opening of the spiral rod (7) by rotational operation of the spiral rod (7) and a lead grasping member (8) provided at a tip end of the reciprocating bar (9), and a holder (2) having a hollow portion which is cylindrical and formed through a tip end. The lead grasping member (8) includes a column-shaped base portion and a plurality of grasping pieces (12) erected upward from the upper peripheral end of the base portion with distant apart in the circumferential direction and upwardly, and guided by the wall of the hollow portion, upon reciprocated. The lead grasping member (8) is configured to be able to be projected for the upper face of the base portion (11) to be exposed from through the tip end opening. A portion of the wall of the hollow portion that guides the plurality of grasping pieces is formed linearly in the reciprocating direction.

### TECHNICAL FIELD

The present invention relates to a cosmetic applicator such as an eyebrow as well as an eyeliner, and particularly, to a cosmetic applicator in which a lead member is easily replaceable by a purchaser himself/herself and which can be repeatedly used without the used container being thrown away.

### BACKGROUND ART

Conventionally, in a cosmetic applicator, for example, a screw-out or push-out type cosmetic pencil or the like, a disposable type one has been in a mainstream, that is, after usage of the lead member, the container that is a plastic product is disposed as a whole, and replaced by a new cosmetic applicator containing a new lead member.

However, in recent years, reducing CO2, as well as eliminating destruction of marine environment caused by plastic products have become a global theme to be solved worldwide, and accordingly, conventionally used plastic products have been gradually and actively replaced by biomass products as well as recycled products.

However, the replacement of plastic products as above mentioned has a problem causing increase in product cost or deterioration in functionality of the product, and thus there has been no rapid progress regarding said replacement motion in the industry.

### PRIOR ART REFERENCE

### Patent Document

Patent Document 1: Japan Patent Application laid open Gazette No. 2003-159119.

### SUMMARY OF THE INVENTION

### Problem to be Solved by the Invention

The present invention was made to solve the aforementioned conventional problem and has for its object to provide a cosmetic applicator, for example, a screw-out or extended-out type cosmetic pencil, whose usage of a lead has finished, and whose lead only can be replaced easily by a new lead, with no necessities of the cosmetic container as a whole to be thrown away and of a new one to be purchased, thereby the cosmetic applicator itself being continuously used.

In other words, the present invention focuses, in particular, on Reduce (Reduction of Discharge) and Reuse (Repeatedly Usable) in the concept of 4R (Refuse, Reduce, Reuse and Recycle). In addition, it is considered that the present invention, which is also superior to ethical consumption (ethical consumption behavior), environmentally friendly, and highly economical, is very advantageous for a user.

Here, it is noted that a lead that is used for an eyebrow or an eyeliner, constitutes a cosmetic pencil lead made of oil and wax as base materials, unlike a hard lead of a stationary. The cosmetic pencil lead is soft, so it is necessary to handle the cosmetic pencil lead carefully. Further, it is very difficult to pull the pencil lead out, with completely removing residual portions of the lead slightly left in a part called a lead chuck for holding the lead.

Conventionally, there has been a problem that a lead whose use has finished remains at a lead chuck, and for example, even if a cosmetic applicator is changed to a type that is loaded with a new lead to be repeatedly usable, it is difficult to remove the remained residual portion completely to effect a complete cleaning. This is a difficulty caused by a structure of the conventional container (the lead chuck being remained in the container).

In addition, conventionally, respectively colored leads are loaded in separate cosmetic applicators, so it has been necessary to purchase respectively colored products in order to purchase products of various colored leads. Therefore, it is necessary to display a number of cosmetic applicators as many as a number of colors at a sales place.

However, according to the present invention, the SKU (inventory management unit) of the cosmetic applicator can be made one by separately selling the cosmetic applicator and refills for the leads, and the inventory risk of the manufacturer can be reduced. Further, the refills for the leads, even if a plurality of the refills are set, are not bulky, and do not occupy large space, so it is possible to use effectively the storefront of a valuable sales shop.

### Means for Solving the Problems

The first invention is characterized in comprising:
a spiral rod formed with a spiral groove on an inner periphery of a column-shaped hollowed portion;
a lead chuck member including a reciprocating bar which has a fitting portion fitted in the spiral groove and which can be reciprocated from through a tip end opening of the spiral rod by rotational operation of the spiral rod, and a lead grasping member provided at a tip end of the reciprocating bar; and
a holder having a wall of a hollow portion which is cylindrical and formed to and through a tip end and in which said lead chuck member is inserted; wherein
said lead grasping member includes a column-shaped base portion and a plurality of grasping pieces erected upwardly from the upper peripheral end of said base portion with distant apart in the circumferential direction and guided by said wall of the hollow portion when said reciprocating bar is reciprocated;
said lead grasping member is configured to be able to be projected for the upper face of said base portion to be exposed from through the tip end opening of said wall of the hollow portion; and a portion of said wall of the hollow portion that guides said plurality of the grasping pieces is formed linearly in said reciprocating direction.

The second invention is characterized in comprising:
a spiral rod provided, on an inner periphery of a hollowed portion thereof, with a spiral groove formed by a spirally elongated projection, the spiral rod being substantially cylindrical;
a lead chuck member including a reciprocating bar which has a fitting portion that is fitted in the spiral groove so that said fitting portion is movable spirally in the spiral groove to be reciprocated in a length direction of the hollowed portion of the spiral rod by rotation of the spiral rod, and a lead grasping member provided at a tip end of the reciprocating bar for attaching the lead; and
a base rod to which said spiral rod is attached rotatably with the axis direction being as the rotation axis, wherein
said lead chuck member is so configured that said lead chuck member is removable from the spiral rod by removing said fitting portion out of said spiral groove, and that after the removal, the lead chuck member attached with a refill lead, is inserted in said spiral rod so that said fitting portion is fitted in the spiral groove by rotational operation of the spiral rod, thereby the lead chuck member being able to be reciprocated in the spiral rod in the length direction of the spiral rod.

### Advantageous Effect of the Invention

According to the cosmetic applicator of the present invention, even in a screw-out or push-out type cosmetic pencil or the like, after a lead has been used, it is not necessary to throw away a cosmetic container as a whole, but if only the used lead is replaced by a refill lead, the cosmetic container can be continuously used without being thrown away, and replacement of the lead can be made easily, residual portions of the lead remained at the lead chuck can be removed by cleaning. Accordingly, the cosmetic container can be repeatedly used without being thrown away, so a burden of a purchaser can be reduced and more over an environmentally friendly cosmetic applicator can be provided. Further, in case of replacement of a lead attachment set attached to a lead holder, the lead holder as a whole can be replaced without the residual of the lead remaining at the lead chuck being touched by hand, and without contaminating the user's hand.

### BRIEF DESCRIPTION OF THE DRAWINGS

FIG. 1 is a configuration explanatory view (1) for explaining a constitution of a first invention.

FIG. 2 is a configuration explanatory view for explaining a constitution of a lead grasping member in the first invention.

FIG. 3 is a configuration explanatory view (2) for explaining a constitution of a first invention.

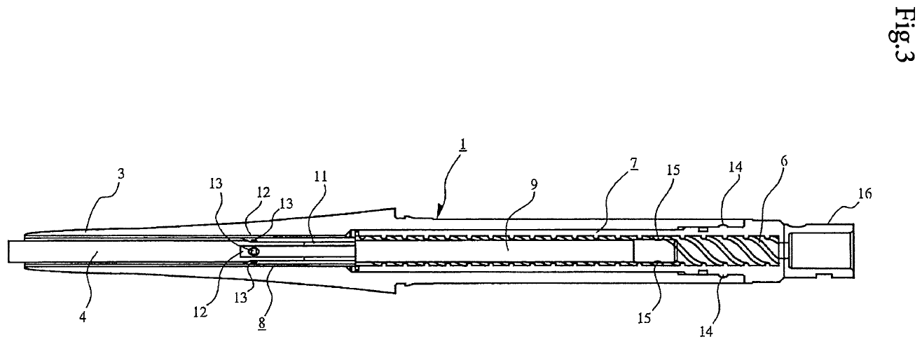

FIG. 4 is a configuration explanatory view (1) for explaining a constitution of a second invention.

FIG. 5 is a configuration explanatory view (2) for explaining the constitution of the second invention.

FIG. 6 is an enlarged view of a main part of the second invention.

FIG. 7 is an explanatory view for explaining a state where a lead chuck member with a lead is on sale separately.

FIG. 8 is a schematic explanatory view explaining, from upper side, a tip opening of a spiral rod.

FIG. 9 is a schematic explanatory view explaining a developed state of an inner periphery of the spiral rod.

FIG. 10 is an explanatory view explaining a state where the lead chuck member is inserted from into the tip opening of a base rod, from upper side.

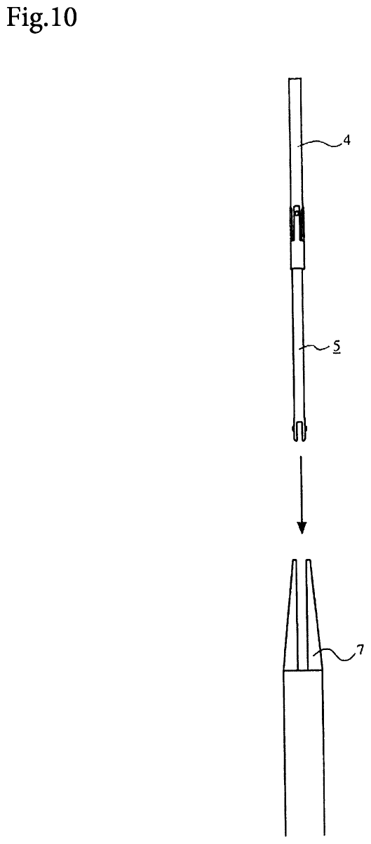

### EMBODIMENTS FOR CARRYING OUT THE INVENTION

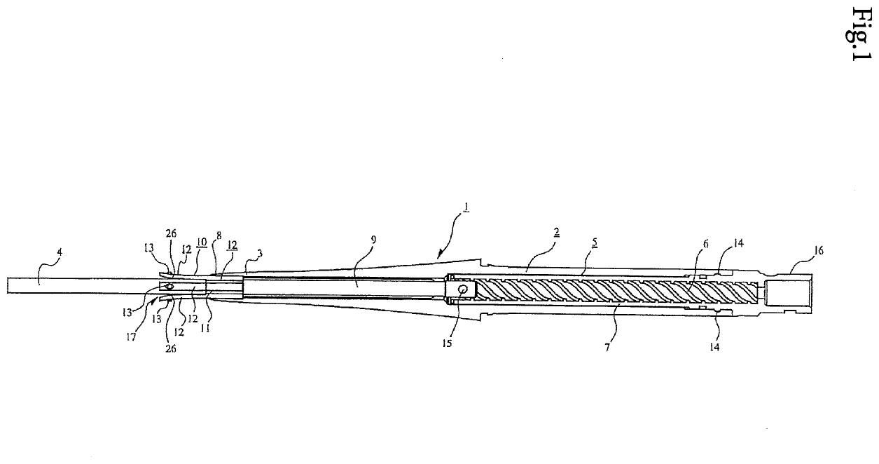

Next, an embodiment of a first invention will be explained. FIG. 1 shows a cosmetic applicator 1 according to the present embodiment. The cosmetic applicator 1 comprises a holder 2 constituted to have a substantially column-shaped hollow portion to be substantially cylindrical. The tip end side of the holder 2 is shaped to form a nib 3 narrowed toward the tip end, so that a proper length of a rod-shaped cosmetic lead 4 may be projected out of the tip opening.

Reference numeral 5 denotes a lead chuck member. The lead chuck member 5 is so configured to be inserted into the hollow portion of the holder 2 from a rear end opening of the holder 2 and to be attached and fixed to the inside of the holder 2.

In more detail, an outer periphery of the rear end of the lead chuck member 5 is formed with a ring-shaped latching projection 14. The latching projection 14 is fitted into a ring-shaped concaved portion provided at an inner periphery of the hollowed portion of the holder 2 so that the lead chuck member 5 is attached to inside the holder 2. Moreover, the lead chuck member 5 is configured by the above mentioned attaching way to be rotatable in the holder 2 about the rotation axis extended in the axial direction of the holder 2.

Here, the lead chuck member 5 is composed in combination of a plurality of parts. Firstly, it includes a spiral rod 7 which has a column-shaped hollow portion and whose outer diameter shape is cylindrical. The inner periphery of the hollowed portion is provided with spiral grooves 6.

A lead grasping member 8 also is included as a part of the lead chuck member 5. The lead grasping member 8 includes a reciprocating bar-shaped member 9 that is loosely fitted in the hollow portion of the spiral rod 7 and movable in the length direction of the spiral rod 7. The reciprocating member 9 is provided, at a tip end thereof, with a lead grasping part 10 for grasping and holding the lead member 4. The lead grasping part 10 includes, for example, a circular column-shaped base portion 11 at a tip end of the reciprocating bar member 9, and a plurality of grasping pieces 12 provided at the outer peripheral end of the tip of the base portion 11 with peripherally distant from each other and upwardly projected.

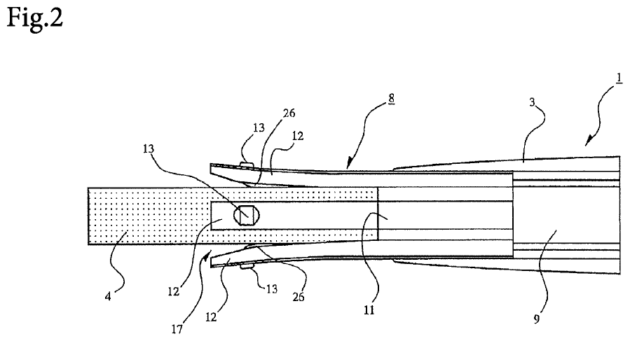

Further, as understood from FIG. 2, the plurality of grasping pieces 12 on the tip end side thereof are configured to bend radially outwardly and shaped to become thin toward tip end with the inner peripheries of the grasping pieces 12 being shaved or cut off. Thus, a guide opening 17 is formed, through which the lead 4 that is soft can be easily inserted into an insertion space surrounded by the plurality of grasping pieces 12 (Refer to FIG. 2). Accordingly, when the soft lead 4 is inserted in the insertion guide opening 17, it is possible to insert it to the upper face of the base portion 11, without being damaged. Further, there are certain spaces between the plurality of the grasping pieces 12 so that the respective grasping pieces 12 are independent from each other, thereby respective grasping pieces 12 having elasticity inside and outside of the insertion space. Accordingly, the structure of the grasping pieces 12 having elasticity, allows the soft lead 4 to be mounted or charged without being damaged.

Incidentally, the respective plurality of grasping pieces 12 is provided on the outer diameter side with outward projections 13, so when the plurality of grasping pieces 12 is retracted into the hollow portion of the holder 2, the projections 13 are brought into contact with the wall of the hollow portion. Then, the respective grasping pieces 12 bend inwardly to fit onto the grasped lead 4 further firmly.

Moreover, the respective grasping pieces 12 are provided projectively, on the inner periphery thereof, with longitudinal ribs 26 extending in the length direction of the grasping pieces 10 in order to prevent the lead from being removed. The lead 4 is further firmly fitted by the ribs 26, and thus the removal of the lead 4 is prevented without the soft lead 4 being damaged.

There are provided at least a pair of guide projections 15 on the outer periphery of the rear end of the reciprocating bar 9, which are fitted in the spiral grooves 6 and displaced along the spiral groves 6, so thereby when the spiral rod 7 is rotated, the reciprocating bar 9 may be moved outside and inside from through the tip opening of the spiral rod 7. These guide projections 15 have elasticity against the spiral grooves 6 such that, even if the spiral rod 7 is continuously rotated at the position where the outer periphery thereof is formed with no spiral groove 6 in the up and down direction, the reciprocating bar-shaped member 9 may not be displaced in the up and down direction of the spiral rod 7. In other words, when the spiral rod 7 is continuously rotated, the guide projections 15 pass over the walls of the spiral grooves 6 to move to the neighboring grooves. Further, even if the rotation of the spiral rod is further continued, the actions that the guide projections 15 pass over the groove walls of the spiral grooves 6 and move to the neighboring grooves 6, are repeated, and there is caused no jumping of the reciprocating bar 9 from the holder 2.

Next, assembling of the cosmetic applicator of the present embodiment and further how to use it will be explained. The lead grasping member 8, with the lead grasping part 10 thereof being made as a tip end and the other end where the guide projections 15 are provided made as the rear end, is inserted from through the tip opening of the spiral rod 7 into the inner space of the spiral rod 7. At this time, the guide projections 15 are fitted in the spiral grooves of the spiral rod 7, and the spiral rod 7 or the lead grasping member 8 is rotated so that the lead grasping member 8 is advanced to the inner most side of the spiral groove 7. The lead chuck member in this state is inserted from through the rear end opening of the holder 2, and the elongated latch projections 14 are fitted in the ring-shaped concave portion of the holder 2. Thus, the cosmetic applicator 1 of the present embodiment is assembled.

By rotating a rear end grip 16 of the spiral rod 7 while the holder 2 is being held, the base portion 11 of the lead grasping part 10 in the lead grasping member 8 can be projected out of the tip end opening of the holder 2 and exposed as shown in FIG. 1. The lead 4 is inserted into the thus projected lead grasping part 10, and then the rear end knob 16 is rotated reversely. Then, the projections 13 are moved in the spiral grooves 6, thereby the lead grasping member 8 being retracted into the holder 2.

And, upon using the cosmetic applicator, the lead 4 is projected out of the tip end opening of the holder 4 by an appropriate length to use an eyebrow or an eyeliner. In a case where, as a result of the use, the lead 4 has become very short, and it is necessary to be replaced, the user holds the holder 2 and rotates the rear end knob 16 of the spiral rod 7, thereby making the base portion 11 of the lead grasping part 10 in the lead grasping member 8 to be projected out of the tip end opening of the holder 2 and exposed. The thus exposed lead 4 whose length has reduced, is taken out. Then, the exposed grasping pieces 12 as well as the face of the base portion 11 are cleaned to remove the residual portions of the lead 4, and a new lead 4 for refill is inserted into the space formed by the grasping pieces 12.

Subsequently, the rear end knob 16 is reversely rotated again, so that the lead 4 is retracted in the holder 4. And, when using, an appropriate length of the lead 4 is projected out and used. Incidentally, the present embodiment can be used for all lead diameters as well as all lead shapes, and further for both of containers with a cap or with no cap.

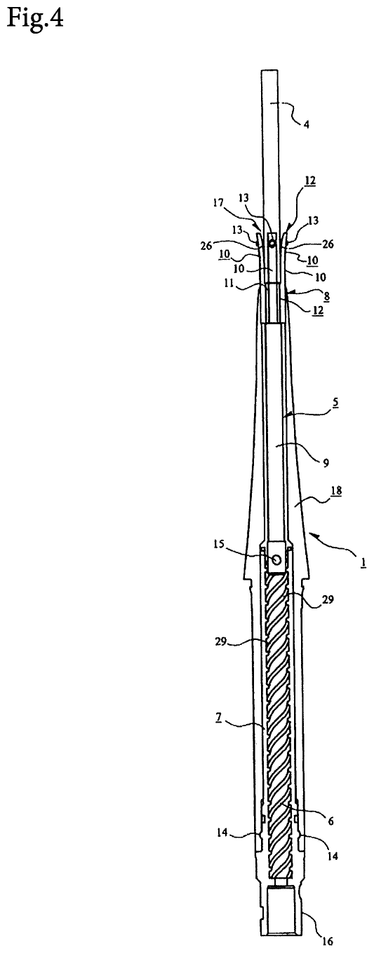

Below, an embodiment of a second invention will be explained. FIG. 4 shows a lead replaceable cosmetic applicator 1 of the present embodiment.

The lead replaceable cosmetic applicator 1 comprises a lead 4, a lead chuck member 5 to which the lead 4 is attached, a spiral rod 7 to which the lead chuck member 5 is attached to be displaceable in the lengthwise direction and a base rod 18 in which the lead chuck member 5 is assembled. They are assembled in combination to compose the fundamental lead replaceable cosmetic applicator 1. Further, the thus assembled fundamental lead replaceable cosmetic applicator 1 is contained in, for example, a cylindrical cosmetic applicator containing body and a cylindrical cap, variously designed and put on the market for sale.

At first, the constitution of the lead chuck member 5 will be explained. The lead chuck member 5 comprises a reciprocating bar 9 which is provided in the spiral rod 7 to be able to be displaced, while being rotated, in the length direction of the spiral rod 7. The reciprocating bar 9 at a tip end thereof is provided with a lead grasping member 8. The lead grasping member 8 comprises a plurality of lead grasping pieces 10 for grasping and holding the lead 4.

The reciprocating bar 9 is substantially cylindrical and provided at the rear end periphery in the length direction thereof with substantially ball-shaped guide projections 15 which are outwardly projected, for example. Meanwhile, it may be so constituted that, in the rear end of the reciprocating bar 9 in the length direction, in a state slightly protruded outside from the outer periphery of the rear end and in a state projected downside, at least two guide pieces shaped as above, are provided with spaced apart in the circumferential direction, and the guide projections 15 shaped as above are provided outsides of the guide pieces. However, without the guide pieces being provided, the guide projections 15 may be provided directly on the outer periphery of the rear end of the reciprocating bar 9.

The spiral rod 7 is formed in a substantially cylindrical hollowed portion with spirally elongated projections 29 projecting from the inner peripheral surface of the hollowed portion toward the axis of the hollowed portion, and formed spirally continuously in the length direction of the hollowed portion.

Starting portions 30 of spiral grooves formed by the spirally elongated projection 29 are not formed at the upper end position of the hollowed portion, but formed at more inner position than the upper end of the hollowed portion. The reason why the spiral groove starting positions 30 are not formed at the upper end position of the hollowed portion, but formed at more inner position than the upper end of the hollowed portion, is that it is possible to insert the rear end side of the reciprocating bar 9 into the hollow portion to reach the more inner position from the upper end portion of the hollowed portion, so that it is possible to improve initial stable holding of the spiral rod 7 by the lead chuck member 5.

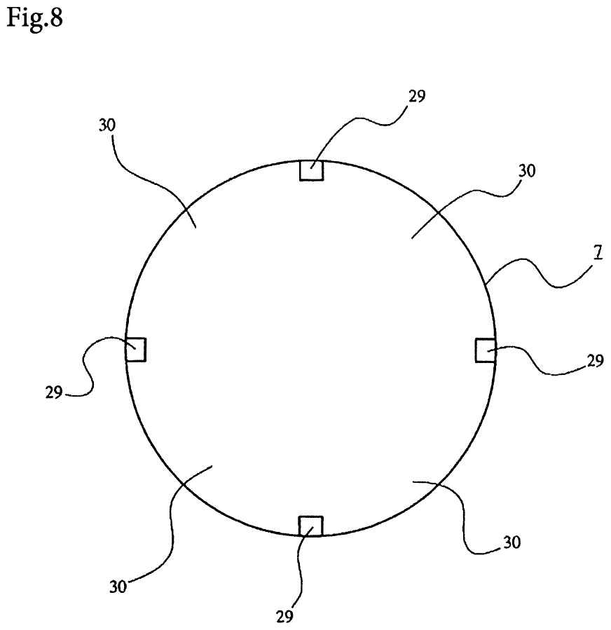

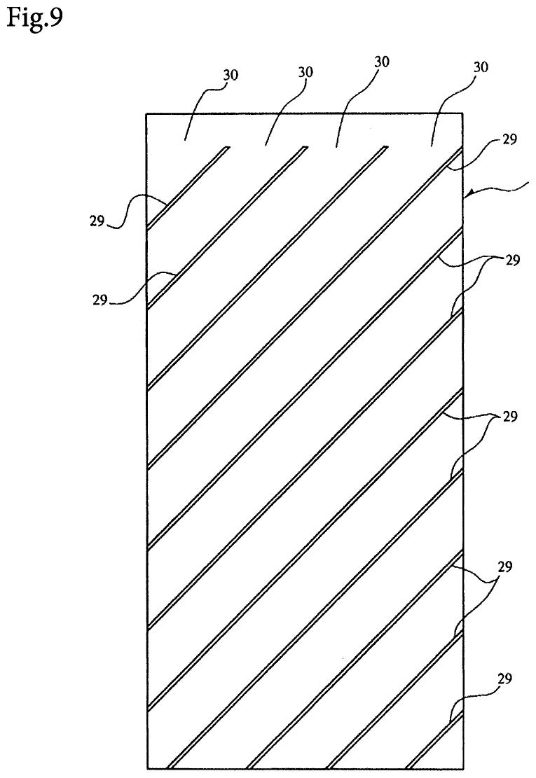

Incidentally, as shown in FIG. 8, four spiral groove starting positions 30 are provided circumferentially distant from each other in the inner periphery of the tip end side hollowed portion of the substantially cylindrical spiral rod 7. FIG. 9 is its developed view. As shown in FIG. 9, there are four spiral groove starting portions 30 in the circumferential direction, respective elongated spiral projections 29 are continuously formed from the respective spiral groove starting portions 50 so that the spiral grooves 6 are elongated spirally to the interior of the spiral rod 7.

In the present embodiment, there are provided four spiral groove starting portions 30. From the starting portions 30, the respective spiral elongated projections 29 are formed continuously. However, the number of the spiral groove starting portions 30 may be two or more.

The smaller the number of the spiral group stating portions 30 is, the smaller the number of the spirally elongated projections 29 continuous thereto is, so widths of the grooves 6 formed by the upper and lower spirally elongated grooves 29 become large. On the contrary, the more the number of the spiral group stating portions 30 is, the more the number of the spiral elongated projections 29 continuous thereto is, so widths of the grooves 6 formed by the upper and lower spiral elongated grooves 29 become small.

According to the present embodiment, as an optimum case where the guide projections 15 provided on the reciprocating bar 9 of the lead chuck member 5 may be spirally rotated smoothly and displaced within the spiral rod 7, there are provided four spiral groove starting portions 30, thereby forming spiral grooves 6 having optimum groove widths.

Accordingly, in a case where a spiral rod 7 having a large length or guide projections 15 having a large size is adopted, the number of the spiral groove starting portions 30 is changed to change groove widths of spiral grooves 6 so that the lead chuck member 5 may be smoothly rotated spirally to displace within the spiral rod 7 in the optimum state.

As to how to attach the lead chuck member 5 to the spiral rod 7, firstly the spiral rod 7 is stood substantially vertically and held. Next, the lead chuck member 5 with the lead grasping piece 10 side being made as tip end and the guide projection 15 side of the rear end of the reciprocating bar 9 being made as rear end, is inserted into the hollow portion of the vertically stood spiral rod 7 from through the tip end opening thereof. Then, the lead chuck member having, for example, at least two or more guide projections 15 drops, by its own gravity, into the hollow portion of the spiral rod 7. And, the guide projections 15 are loosely fitted in the spiral groove starting portions 30 and latched therein.

In a state where the guide projections 15 are latched in the spiral groove starting portions 30, if the spiral rod 7 is rotated using the rear end knob 16 of the spiral rod 7, the guide projections 15 drop spirally smoothly in along the spiral grooves 6 continuous to the spiral groove starting portions 30 to advance in the interior of the spiral rod 7. And, by further rotating the spiral rod 7, it is possible to advance the reciprocating bar 9 and the lead chuck member 15 to the most interior side of the spiral groove 7.

That is to say, by rotating the spiral rod 7, the guide projections 15 provided on the reciprocating bar 9, loosely fitted in the spiral grooves 6 provided on the inner periphery of the hollowed portion of the spiral rod 7, fall therealong to displace in the interior side of the hollowed portion. By the spirally falling displacement of the guide projections 15 in along the spiral grooves 6, the reciprocating bar 9 and consequently the lead chuck member 5 can be displaced, while being rotated, to the interior side of the spiral rod 7.

Incidentally, as above described, it is preferable that the guide projections 15 which are substantially spherically projected are provided at least two on the circular rear end of the reciprocating bar 9 or on the guide pieces provided on the rear end to oppose to each other, but a plurality of the projections more than two may be provided also.

Further, the tip end opening of the hollowed portion of the spiral rod 7 is so configured that the guide projections 15 may be easily inserted. In other words, the tip end opening of the hollowed portion of the spiral rod 7 is so configured to be provided with guide surfaces slightly widen outwardly so that the guide projections 15 may be inserted in the spirally elongated projections 29, and by the presence of the guide surfaces the rear end of the reciprocating bar 9 can be easily inserted within the hollowed portion of the spiral rod 7.

Thus, if the spiral rod 7, in the state where the guide projections 15 are loosely fitted and latched in the spiral groove staring portions 30, is rotated, the guide projections 15 slide on the lower spiral elongated projections 29 to be displaced in the spiral grooves 6.

In other words, with use of the rear end knob 16 provided at the rear end portion of the spiral rod 7 or the cosmetic applicator body that is so constituted to be operatively associated with the rear end knob 16, the spiral rod 7 is rotated, thereby it being possible that the lead chuck member having the reciprocating bar 9 is inserted into the hollow portion of the spiral rod 7 and is retracted to the tip end side of the spiral rod 7.

As explained above, a constitution that the lead chuck member 5 is able to be inserted into the spiral rod 7 and receded therefrom, is achieved by a constitution that the guide projections 15, for example, at least two, provided on the rear end portion of the reciprocating bar 9 are able to be loosely fitted, latched and spirally slid in the spiral groove starting portions 30 and the spiral grooves 6 continuous thereto, provided on the hollowed portion 30 of the spiral rod 7.

Further, when the subject matter is used as an eyebrow or an eyeliner, the lead chuck member 5 attached with the lead 4 is prevented by the upper and lower spirally elongated projections 29 from being displaced by itself in the length direction of the spiral rod 7, since the guide projections 15 are retained in the spiral grooves formed by the upper spirally elongated groove 29 and the lower spirally elongated groove 29.

Next, a way for inserting and attaching the spiral rod 7 attached with the lead chuck member 15 attached with the lead 4, into the hollow portion of the base rod 18, will be explained.

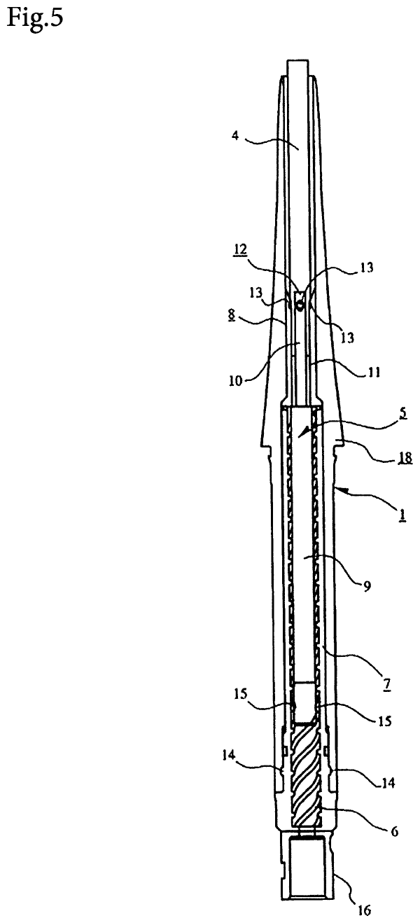

Referring to FIG. 4 and FIG. 5, reference numeral 14 denotes a ring-shaped elongated outward latch projection provided on the outer periphery of rear end side of the spiral rod 7. The spiral rod 7 is pushed to be inserted into the hollow portion of the base rod 18 from through rear end opening of the base rod 18 and the ring-shaped elongated outward latch projection 14 is fitted in the ring-shaped concave portion formed in the hollowed portion of the base rod 18, thereby the base rod 18 and the spiral rod 7 are integrated and the spiral rod 7 can not be pulled out easily from the base rod 18. And, in a state where the outer periphery of the base rod 18 is held, the spiral rod 7 can be easily rotated about the rotation axis extending in the axial direction of the base rod 18 in the state where the latch elongated projection 14 and the ring-shaped concave portion provided in the inner periphery of the hollow portion of the base rod 18 are fitted. Thus, the lead interchangeable cosmetic applicator 1 as a fundamental component according to the present embodiment is assembled. Thus, assembled component is contained in a cylindrical body and a cylindrical cap to form the lead interchangeable cosmetic applicator 1 to which various kinds of designs are applied. And, the cosmetic applicator body with the spiral rod 7 is configured to be operatively associated with the rear end knob 16, so that rotation of the cosmetic applicator body causes rotation of the spiral rod 7.

Here, after having assembled the lead replaceable cosmetic applicator 1 according to the present embodiment, while the outer periphery of the base rod 18 exposed on the tip end side is being held, the cosmetic applicator body is rotated. Then, the spiral rod 7 is also rotated, and by this rotation, the lead 4 chucked by the lead chuck member 5 can be smoothly protruded out from through the tip end opening of the base rod 18 or retracted into the interior of the base rod 18.

The present embodiment is so constituted that when the spiral rod 7 is continuously rotated the lead chuck member 5 itself does not jump out from through the tip end opening of the base rod 18, as shown in FIG. 5. That is to say, the guide projections 15 are retained at the portion of the spiral groove starting portions 30, and at this portion of the spiral groove starting portions 30 the guide projections 15 are repeatedly rotated. Thus, the guide projections 15 and the lead chuck member 5 are repeatedly rotated only, but do not jump out from through the tip end opening of the base rod 18.

Meanwhile, the lead grasping pieces 12 composing the lead chuck member 5 have a cylindrical base portion 11 on the tip end side of the reciprocating bar 9, and from the outer peripheral end of the base portion 11, a plurality of the lead grasping pieces 10 are formed upwardly with spaced apart in the circumferential direction.

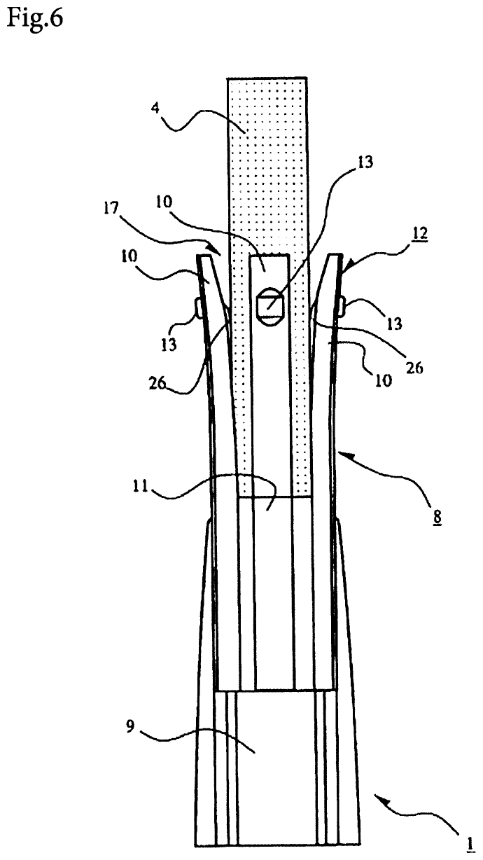

Further, as understood from FIG. 4, FIG. 5 and FIG. 6, the plurality of grasping pieces 10 are, on the tip end side thereof, configured to bend radially outwardly and shaped to become thin toward tip end with the inner periphery of the grasping pieces 10 being shaved or cut off. Thus, a guide opening 17 is formed through which the soft lead 4 can be easily inserted into a lead insertion space surrounded by the plurality of grasping pieces 10 (Refer to FIG. 4, FIG. 5 and FIG. 6).

Accordingly, when the soft lead 4 is inserted in the insertion guide opening 17, the lead 4 can be inserted until it is brought in contact with the upper face of the base portion 11 without being damaged. Further, there are predetermined intervals between the plurality of the grasping pieces 10 so that the respective grasping pieces 10 are erected independently from each other.

And, the grasping pieces 10 have elasticity to bend easily toward inside and outside of the insertion space. The structure of the grasping pieces 10 having elasticity, allows the soft lead 4 to be mounted or charged without being damaged.

The plurality of grasping pieces 10 on the outer peripheral surface side is provided respectively with outward projections 13, so when the plurality of grasping pieces 10 is advanced into the hollow portion of the base member 18, the projections 13 are brought into contact with the wall of the hollow portion. Then, the respective grasping pieces 10 bend inwardly to fit onto the grasped lead 4 further firmly.

Moreover, the respective grasping pieces 10 are provided projectively, on the outer periphery thereof, with longitudinal ribs 26 extending in the length direction of the grasping pieces 10, for example, in order to prevent the lead from being removed. The lead 4 is further firmly fitted by the ribs 26, and thus the removal of the lead 4 is prevented without the soft lead 4 being damaged. Meanwhile, the above explained projections 13 and the ribs 16 may not be provided. The lead grasping member 8 has been provided with lead 4 previously.

And, as described previously, after having inserted the lead chuck member 5 attached with the lead 4 into the spiral rod 7, the spiral rod 7 with this lead chuck member 5 is fitted into the inner space of the base rod 18 to make the fundamental lead replaceable cosmetic applicator, and then to the fundamental lead replaceable applicator, the cosmetic applicator body and the cylindrical cap are attached, thereby finishing assembling of the lead replaceable cosmetic applicator applied with various kinds of designs.

A way of replacing the lead 4 in the cosmetic applicator according to the present embodiment will be explained. As shown in FIG. 4, the lead replaceable cosmetic applicator 1 is held with stood substantially vertically. Then, the cosmetic applicator body constituted to be operatively associated with the rear end knob 16 of the spiral rod 7 is rotated. Thus, it is possible for the base portion 11 of the lead grasping pieces 12 in the lead grasping member 8 to be protruded out from the tip end opening portion of the base rod 18 until being exposed, as shown in FIG. 4.

Meanwhile, after the base portion 11 of the lead grasping pieces 12 having been exposed to the tip end opening portion of the base rod, even if the cosmetic applicator body is further rotated, the base portion 11 of the lead grasping pieces 12 does not move out further. This is because the guide projections 15 are rotated and slid only at the spiral groove starting portion 30 but do not move further upward in the inner space of the spiral rod 7. Thus, the lead 4 whose length has been reduced is protruded and exposed as described above, is removed out from the lead grasping pieces 12. After the shortened lead 4 has been removed, a new lead 4 for replacement is inserted. The new lead 4 can be inserted smoothly within the lead grasping pieces 12.

The lead 4 is inserted in the lead grasping pieces 12 and attached thereto, as described above, and then the cosmetic applicator body operatively associated with the rear end knob 16 is reversely rotated. Then, the guide projections 15 is displaced along in the spiral grooves 6, and the lead grasping member 8 is receded in the most interior side of the spiral groove 7. And, upon using, the lead 4 is protruded out from through the tip end opening of the base rod 18. Thus, the applicator can be used as an eyebrow or an eyeliner.

During using the applicator as an eyebrow or an eyeliner, the lead chuck member 5 with the lead 4 does not make any fluctuating movement in the longitudinal direction of the lead chuck member 5. If the lead 4 is moved during used, it is impossible to apply cosmetics.

By the way, in a case where the lead 4 has been used and become quite small in length and its replacement is required, and in a case where only the lead 4 is replaced, the present cosmetic applicator is held substantially vertically, as described herein before, and length of the lead grasping pieces 12 to the base portion 11 of the lead grasping member 8 is made to be exposed out of the tip end opening of the base rod 18, as shown in FIG. 4. Thus, the shortened lead 4 in the lead grasping pieces 12 has been removed away. It has been necessary, however, that when the lead 4 is removed, it has been necessary to perform cleaning of the lead grasping pieces 12 as well as upper faces of the base portion 11 to remove residual portions of the lead 4. The present embodiment has solved such a problem. In other words, in the above, an example by which only the lead 4 is replaced was explained. However, the present embodiment is not limited thereto.

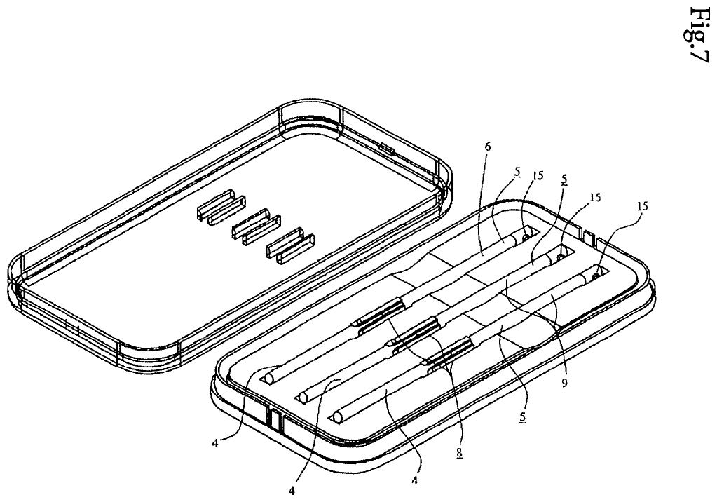

As shown in FIG. 7, refills for lead 4 are put on the market in the state where the lead 4 has been previously attached to the lead chuck member 5. This is because selling lead 4 only so that user may conduct replacement of the lead 4, is considered to impose heavy burden on the user.

A first problem is to contaminate user's fingers. It is necessary to grasp a refill lead 4 directly by hand and perform replacement operation. Secondly, after having removed the shortened lead 4, it is necessary to conduct cleaning for the lead grasping pieces and the upper surface of the base portion 11 and remove residual portions of the lead 4. This cleaning has to be done by user himself/herself. And, for removing the residual portion of the lead 4, any tool, such as a toothpick, has to be used. But, such a tool, as toothpick, might break the lead grasping pieces 12.

Accordingly, refill sets each containing refills of lead 4 previously attached to the lead chuck member 5 are prepared and sold separately. And, the lead replaceable cosmetic applicator 1 of the present embodiment is so configured that even user may conduct easily removal operation of the lead chuck member 5 attached with the shortened lead 4, and conduct replacing it easily by a lead chuck member 5 attached with a refill lead 4.

Away of the replacement will be explained. Firstly, the lead replaceable cosmetic applicator 1 is stood substantially vertically and held. As a matter of course, the tip end opening of the base rod 18 as a pen nib is on the tip end side.

The lead chuck member 5 having the shortened lead 4 is displaced, by rotating operation, spirally to a position where the guide projections 15 are located at the spiral groove starting portions 30 from a position of the spirally elongated projections 29. Thus, the guide projections 15 are released from the spiral grooves 6 formed by the upper and lower spirally elongated projections 29. That is to say, there becomes no upper spirally elongated groove above the guide projections 15, so removing the guide projections 15 upwardly from the spiral rod 7 can not be prevented. Accordingly, the lead chuck member 5 may be easily removed out from the spiral rod 7.

Accordingly, the lead chuck member 5 itself which holds the shortened lead 4 can be removed out of the spiral groove 7, and therefore from the base rod 18, without directly touching with the shortened lead 4.

Thus, there is no possibility that user might touch the lead 4 and get his/her finger(s) dirty. Further, the lead chuck member 5 is replaceable, so it is not completely necessary to conduct cleaning for removing residual portion of the lead 4 out of the lead chuck member 8.

Here, regarding set sale of only refill leads 4 and set sale of lead chuck members 5 each attached with refill lead 4, since the lead chuck member 5 itself can be manufactured and sold quite less expensively, both of the set sales are not so different in selling price.

Consumer takes out a lead chuck member 5 attached with a refill lead 4, sold separately and previously purchased. While holding a lead replaceable cosmetic applicator 1 substantially vertically, the consumer drops the lead chuck member attached with a refill lead 4, into the applicator from the tip opening of the base rod 18 to be inserted thereinto. This insertion of the base rod is made from a side where guide projections 15 are formed (Refer to FIG. 7).

Here, the inner peripheral surface of the hollowed portion of the base rod 18 is formed with a plurality of guide grooves distant apart from each other in peripheral direction and extended in length direction so that the lead chuck member drops smoothly, thereby the guide projections 15 being advanced into the most inside of the base rod 18 along the guide grooves to agree with the tip opening of the spiral rod 7 securely.

It may be so configured that inner diameter of the hollowed portion of the base rod 18 is made large so that the guide projections 15 are not interfered with the inner peripheral surface of the hollow portion of the base rod 18, but in this case, it is difficult for the rear end of the reciprocating bar 9 to be brought into agreement with the tip opening of the spiral rod 7 smoothly. This is because if the hole of the hollowed portion is made large, the rear end of the reciprocating bar 9 would sway and not agree with the tip opening of the spiral rod 7. This is the reason why the guide grooves are formed on the inner periphery of the hollowed portion.

Upon inserted, the lead chuck member 5 to which the refill lead 4 is attached, drops by its own gravity in the hollowed portion of the base rod 18 along the guide grooves and passes therethrough to enter into the hollowed portion of the spiral rod 7, so that the guide projections 15 are brought into loose fit in the spiral rod 7 and latched. Thus, the lead chuck member 5 to which the refill lead 4 is attached, is held in the spiral rod 7.

In such a state, if the spiral rod 7 is rotated, the guide projections 15 of the lead chuck member 5, while rotating, spirally slide and move smoothly in the spiral grooves 6 continuous to the spiral groove starting portion 30, and move to the most interior side of the spiral rod 7 while spirally rotating.

If the lead chuck member 5 is received sufficiently in the base rod 18, a lead replaceable cosmetic applicator attached with a new lead 4 is completed and can be used like a new applicator.

### EXPLANATION OF REFERENCES

1 cosmetic applicator
2 holder
3 nib
4 lead
5 lead chuck member
6 spiral groove
7 spiral rod
8 lead grasping member
9 reciprocating bar
10 lead grasping portion
11 base portion
12 grasping piece
13 projection
14 latch projection
15 guide projection
16 rear end knob
18 base rod
26 rib
29 spirally elongated projection
30 spiral groove starting portion

What is claimed is:

1. A cosmetic applicator comprising:
a spiral rod formed with a spiral groove on an inner periphery of a column-shaped hollowed portion;
a lead chuck member including a reciprocating bar which has a fitting portion fitted in the spiral groove and which can be reciprocated from through a tip end opening of the spiral rod by rotational operation of the spiral rod, and a lead grasping member provided at a tip end of the reciprocating bar; and
a holder having a wall of a hollow portion which is cylindrical and formed to and through a tip end and in which said lead chuck member is inserted; wherein
said lead grasping member includes a column-shaped base portion and a plurality of grasping pieces erected upwardly from the upper peripheral end of said base portion with distant apart in the circumferential direction and guided by said wall of the hollow portion when said reciprocating bar is reciprocated;
said lead grasping member is configured to be able to be projected for the upper face of said base portion to be exposed from through the tip end opening of said wall of the hollow portion;
a portion of said wall of the hollow portion that guides said plurality of the grasping pieces is formed linearly in said reciprocating direction.

2. The cosmetic applicator according to claim 1, wherein said lead grasping pieces are formed on the outer side surfaces with projections toward outside.

3. A lead exchangeable cosmetic applicator comprising:
a spiral rod provided, on an inner periphery of a hollowed portion thereof, with a spiral groove formed by a spirally elongated projection, the spiral rod being substantially cylindrical;
a lead chuck member including a reciprocating bar which has a fitting portion that is fitted in the spiral groove so that said fitting portion is movable spirally in the spiral groove to be reciprocated in a length direction of the hollowed portion of the spiral rod by rotation of the spiral rod, and a lead grasping member provided at a tip end of the reciprocating bar for attaching the lead; and
a base rod to which said spiral rod is attached rotatably with the axis direction being as the rotation axis, wherein
said lead chuck member is so configured that said lead chuck member is removable from the spiral rod by removing said fitting portion out of said spiral groove, and that after the removal, the lead chuck member attached with a refill lead, is inserted in said spiral rod so that said fitting portion is fitted in the spiral groove by rotational operation of the spiral rod, thereby the lead chuck member being able to be reciprocated in the spiral rod in the length direction of the spiral rod.

4. The lead exchangeable cosmetic applicator according to claim 3, wherein the configuration that the lead chuck member is able to be reciprocated in the spiral rod in the length direction of the spiral rod, is a configuration that said spiral rod, at an upper side of the hollowed portion thereof, is formed with a spiral groove starting portion, in which spiral groove starting portion said fitting portion is loosely fitted and latched, so that said fitting portion is able to be fitted in the spiral groove to be advanced in the most interior side of the spiral rod by rotating the spiral rod, and receded back at the tip end side of said spiral groove by reversely rotating the spiral rod.

5. The lead exchangeable cosmetic applicator according to claim 4, wherein
the configuration that the lead chuck member is able to be reciprocated in the spiral rod in the length direction of the spiral rod, is a configuration that said spiral rod is so held substantially vertically for a tip end opening of said spiral rode to be directed upward, the lead chuck member being inserted from the guide projection side of said lead chuck member in said tip end opening so that said fitting portion is loosely fitted and latched in said spiral groove starting portion, and the spiral rod is rotated.
